(19) United States
(12) Patent Application Publication
(10) Pub. No.: US 2022/0183976 A1
(43) Pub. Date: Jun. 16, 2022

(54) COMPOSITIONS OF DISPERSED PHASE FOR PREPARATION OF APIXABAN-LOADED MICROSPHERES AND BIOCOMPATIBLE POLYMER-BASED APIXABAN-LOADED MICROSPHERES PREPARED THEREFROM

(71) Applicant: HLB PHARMACEUTICAL CO., LTD., Gyeonggi-do (KR)

(72) Inventors: Dooyong JEONG, Gyeonggi-do (KR); Sang-Hwi LEE, Gyeonggi-do (KR); Mijung KIM, Gyeonggi-do (KR); Ye-Ji KIM, Ulsan (KR); Jae Hyung PARK, Seoul (KR)

(73) Assignee: HLB PHARMACEUTICAL CO., LTD., Gyeonggi-do (KR)

(21) Appl. No.: 17/598,491

(22) PCT Filed: Mar. 20, 2020
(86) PCT No.: PCT/KR2020/003876

§ 371 Date: Sep. 27, 2021

(30) Foreign Application Priority Data
Mar. 27, 2019 (KR) 10-2019-0035354

(51) Int. Cl. A61K 9/16; A61K 31/4545

(52) U.S. Cl. CPC A61K 9/1647; A61K 9/1617; A61K 31/4545

(57) ABSTRACT

The present disclosure relates to a composition of dispersed phase for the preparation of Apixaban-loaded microspheres and biocompatible polymer-based Apixaban-loaded microspheres prepared therefrom. Specifically, the present disclosure relates to a composition of dispersed phase for the preparation of Apixaban-loaded microspheres, where the composition includes i) Apixaban or a pharmaceutically acceptable salt thereof; ii) a biocompatible polymer; iii) a fatty acid or triglyceride; and iv) a halogen organic solvent; and biocompatible polymer-based Apixaban-loaded microspheres. The composition of dispersed phase for the preparation of Apixaban-loaded microspheres shows excellent stability and thus may be useful for the preparation of Apixaban-loaded microspheres. Additionally, the biocompatible polymer-based Apixaban-loaded microspheres may be contained in pharmaceutical compositions to be used as a therapeutic agent, because the Apixaban may be stably encapsulated therein in high contents and the initial drug release thereof may be suppressed.

### CROSS-REFERENCE TO RELATED APPLICATIONS

This application is the U.S. National Phase of PCT/KR2020/003876 filed Mar. 20, 2020, which claims priority to KR 10-2019-0035354 filed Mar. 27, 2019, the entire disclosures of which are incorporated by reference herein.

### TECHNICAL FIELD

The present disclosure relates to a composition of dispersed phase for the preparation of Apixaban-loaded microspheres and biocompatible polymer-based Apixaban-loaded microspheres prepared therefrom. Specifically, the present disclosure relates to a composition of dispersed phase for the preparation of Apixaban-loaded microspheres, including i) Apixaban or a pharmaceutically acceptable salt thereof; ii) a biocompatible polymer; iii) a fatty acid or triglyceride; and iv) a halogen organic solvent, and biocompatible polymer-based Apixaban-loaded microspheres prepared therefrom.

### BACKGROUND

Drugs which are continuously administered to patients have been developed as sustained release injections so as to enhance the convenience of drug administration to patients. For example, with respect to liposomes or nanoparticles, drug release is completed in vivo within 1 week, and with respect to microspheres composed of synthetic polymers, such as polylactic-co-glycolic acid (PLGA), polylactic acid (PLA), and polycaprolactone (PCL), drug release is completed within 1 week to 18 months after injection in vivo depending on the composition of the polymers, the form of the microparticles, the solubility of the drug, and the method of preparing the microspheres. As such, the long-acting injectable formulations have the advantage of maintaining the in vivo drug concentration within the effective range for a long time when administered to patients, and thus have been mainly developed for the purpose of treating diseases such as dementia, diabetes, Parkinson's disease, etc., which require a continuous drug administration. Additionally, they have been developed for the purpose of altering the route of administration, reducing side effects of the drug, and providing local drug treatments, etc., in addition to increasing the convenience of drug administration (reducing dose frequency) by continuous maintenance of the in vivo effective concentration of the drug.

In order to develop such microsphere formulations using PLGA, PLA, or PCL, the physical properties of the drug, the dosage of the drug, the physicochemical compatibility of the drug and the polymer, and the solubility of the drug in an organic solvent phase should be considered. Even when all of the above factors are considered, the drug release patterns of the formulations can be affected by the types of preparation method and the process parameters.

Apixaban is an active pharmaceutical ingredient which is administered for the purpose of preventing venous thromboembolism in adult patients who have had a hip or knee replacement surgery, reducing the risk of stroke and systemic embolism in patients with nonvalvular atrial fibrillation, and treating deep vein thrombosis and pulmonary embolism and reducing the risk of recurrence thereof. The recommended duration of administration for each indication is as follows: 32 days to 38 days for a hip replacement, 10 days to 14 days for a knee replacement, a continuous administration is recommended for patients with nonvalvular atrial fibrillation in order to prevent stroke and systemic embolism, 7 days for the treatment of deep vein thrombosis and pulmonary embolism, and a long-term drug administration for more than 6 months is recommended for reducing the risk of recurrence of deep vein thrombosis and pulmonary embolism. Accordingly, in the case of Apixaban for which a continuous administration is recommended according to each indication as described above, although Apixaban may have great advantages in terms of increasing patient convenience when developed into a long-acting injection, it has never been developed in microsphere formulations, which can be prepared using polymers such as PLGA, PLA, or PCL. This is because if Apixaban is dissolved in a halogen organic solvent (i.e., a solvent generally used for the preparation of microspheres), the drug is reprecipitated in the solution over time and thus cannot be produced in a large scale for use in industrial applications, and also, the drug rapidly forms crystals upon exposure or dispersion to an aqueous phase.

Generally, the initial burst release is recognized as the most serious problem of the PLGA, PLA or PCL based microspheres. The biocompatible polymer-based microspheres are administered to patients mainly via a subcutaneous or intramuscular injection, and local bleeding can often occur depending on the injection site and depth of penetration of the needle. Apixaban selectively inhibits the factor Xa in the blood coagulation step, thereby preventing the formation of blood clots. Thus, when Apixaban is released from the microspheres by an initial burst release, local lumps can be formed due to bleeding around the injection site. Therefore, in order to develop injectable Apixaban microspheres, there is a need to reduce the amount of drug released within the initial 30 minutes of injection (hereinafter, referred to as initial drug release or initial burst release) at which hemostasis is completed around the injection site. The initial burst release occurs due to the drug diffusion which is caused by the difference in osmotic pressure between the inside of the microspheres and the outer aqueous phase, and the micro-water channels which is formed in the inner structure of the microspheres. In order to resolve this problem, a pore closing technique (Journal of Controlled Release 112 (2006) 167-174) is typically used. However, in the case of Apixaban, a porous microsphere surface may not be formed in the microsphere formation step, and thus, the rapid initial release of Apixaban cannot be inhibited by the technique above.

### SUMMARY

The present inventors have studied the composition of dispersed phase for the preparation of Apixaban-loaded microspheres which can exhibit a stable drug encapsulation and decreased initial burst release. Also, the reprecipitation of Apixaban is prevented in the dispersed phase of present disclosure.

As a result, when a fatty acid or triglyceride is added to the composition of dispersed phase for the preparation of Apixaban-loaded microspheres, it was confirmed that the composition exhibits excellent stability, Apixaban is stably encapsulated into the microspheres in high contents, and that the initial drug release of Apixaban from the biocompatible polymer-based microspheres prepared therefrom is reduced, thereby completing the present disclosure.

An object of the present disclosure is to provide a composition of dispersed phase for the preparation of Apixaban-loaded microspheres, including: i) Apixaban or a pharmaceutically acceptable salt thereof; a biocompatible polymer; iii) a fatty acid or triglyceride; and iv) a halogen organic solvent.

Another object of the present disclosure is to provide a biocompatible polymer-based Apixaban-loaded microsphere.

Still another object of the present disclosure is to provide a pharmaceutical composition including the biocompatible polymer-based Apixaban-loaded microsphere.

The composition of dispersed phase for the preparation of Apixaban-loaded microspheres shows excellent stability and thus can be useful for the preparation of Apixaban-loaded microspheres. Additionally, the biocompatible polymer-based Apixaban-loaded microspheres can be contained in pharmaceutical compositions to be used as a therapeutic agent, because the apixaban can be stably encapsulated therein in high contents and the initial drug release thereof can be suppressed.

### BRIEF DESCRIPTION OF THE DRAWINGS

FIG. 1 is an image taken with a digital camera after adding Apixaban to each of ethyl acetate, ethyl formate, methyl propionate, and ethanol, which are non-halogen organic solvents, followed by stirring.

FIG. 2 shows images taken with a digital camera immediately and 12 hours after dissolving Apixaban in dichloromethane.

FIG. 3 shows images of Apixaban crystals formed 12 hours after dissolving Apixaban in dichloromethane observed under an optical microscope.

FIG. 4 shows images taken with a digital camera at 0, 15, and 30 minutes after dissolving Apixaban and polymer in dichloromethane.

FIG. 5 shows images of polymer-Apixaban precipitates formed after dissolving Apixaban and polymers in dichloromethane observed under an optical microscope.

FIG. 6 shows images taken with a digital camera at 0 minutes and 6 hours after dissolving Apixaban, fatty acid (stearic acid), and polymers in dichloromethane.

FIG. 7 shows images taken with a digital camera at 0 minutes and 6 hours after dissolving Apixaban, fatty acid (lauric acid), and polymers in dichloromethane.

FIG. 8 shows images taken with a digital camera at 0, 15, 30, and 45 minutes after dissolving Apixaban and polymers in dichloromethane.

FIG. 9 shows images illustrating the stability of the compositions of dispersed phase for the preparation of Apixaban-loaded microspheres according to the molar ratio of Apixaban to fatty acid (stearic acid).

FIG. 10 shows images illustrating the stability of the compositions of dispersed phase for the preparation of Apixaban-loaded microspheres according to the molar ratio of Apixaban to fatty acid (lauric acid).

FIG. 11 is an optical microscopic image of Apixaban-loaded microspheres prepared by a solvent evaporation method using a general composition of dispersed phase (drug+polymer+halogen organic solvent) for the preparation of Apixaban-loaded microspheres.

FIG. 12 is an optical microscopic image of Apixaban-loaded microspheres prepared by a microfluidic method using a general composition of dispersed phase (drug+polymer+halogen organic solvent) for the preparation of Apixaban-loaded microspheres.

FIG. 13 is an optical microscopic image of the Apixaban-loaded microspheres (Example 1) prepared in Experimental Example 5-1.

FIG. 14 is an optical microscopic image of the Apixaban-loaded microspheres (Example 2) prepared in Experimental Example 5-2.

FIG. 15 is an optical microscopic image of the Apixaban-loaded microspheres (Example 3) prepared in Experimental Example 5-3.

FIG. 16 is an optical microscopic image of the Apixaban-loaded microspheres (Example 4) prepared in Experimental Example 5-4.

FIG. 17 is an optical microscopic image of the Apixaban-loaded microspheres (Example 5) prepared in Experimental Example 5-5.

FIG. 18 is an optical microscopic image of the Apixaban-loaded microspheres (Example 6) prepared in Experimental Example 5-6.

FIG. 19 is an optical microscopic image of the Apixaban-loaded microspheres (Example 7) prepared in Experimental Example 5-7.

FIG. 20 is a graph showing the drug release rate of Apixaban-loaded microspheres (Examples 1 to 3) prepared in Experimental Example 5.

FIG. 21 is a graph showing the drug release rate of Apixaban-loaded microspheres (Examples 2, 5, and 6) prepared in Experimental Example 5.

### DETAILED DESCRIPTION

In order to achieve the objects, one aspect of the present disclosure provides a composition of dispersed phase for the preparation of Apixaban-loaded microspheres, including: i) Apixaban or a pharmaceutically acceptable salt thereof; ii) a biocompatible polymer; iii) a fatty acid or triglyceride; and iv) a halogen organic solvent.

Specifically, one aspect of the present disclosure may provide a composition of dispersed phase for the preparation of Apixaban-loaded microsphere in the form of an injection for sustained release, which includes the constituents above.

As used herein, the term “Apixaban” refers to a compound having the structure of Chemical Formula 1. Apixaban has three amides in the structure and has an intrinsic dipole structure of the amides. Thus, Apixaban can form intermolecular hydrogen bonds composed of N—H . . . O, and thus can form co-precipitates in a suitable solvent when a proton donor or a proton accepter is present, or can also form intermolecular hydrogen bonds between Apixaban molecules. Accordingly, even when only Apixaban is dissolved, crystals may be formed after a certain period of time.

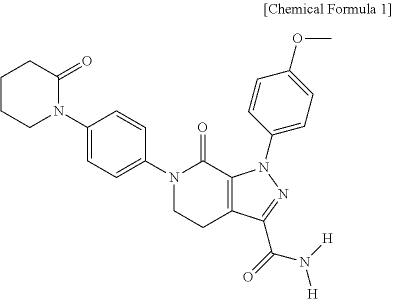 [SMILES: COc1ccc(-n2nc(C(N)=O)c3c2C(=O)N(c2ccc(N4CCCCC4=O)cc2)CC3)cc1]

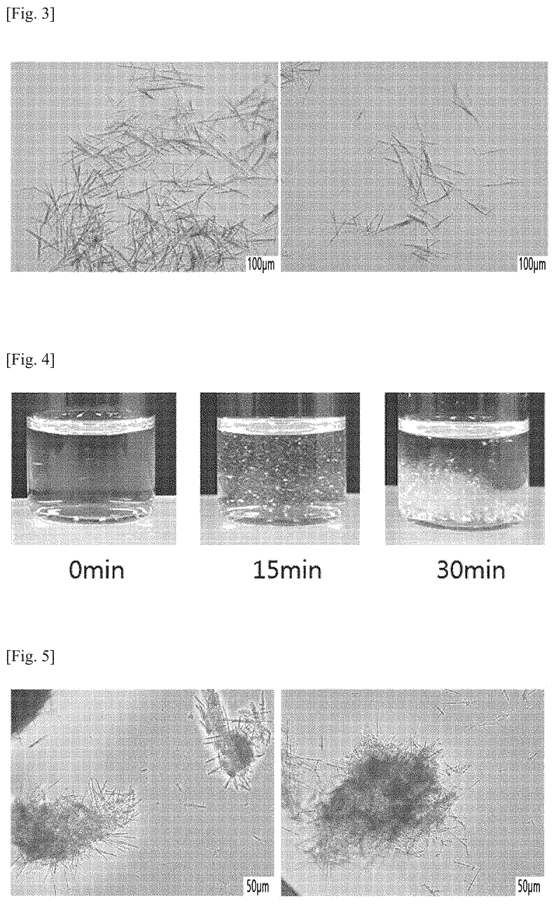

In one specific embodiment of the present disclosure, when Apixaban was dissolved in dichloromethane, which is an organic solvent most commonly used in the preparation of PLGA, PLA, or PCL-based microspheres, it was confirmed that the solubility of Apixaban in dichloromethane was exceedingly high that no crystals of Apixaban were observed immediately after dissolution, but after 12 hours of dissolution, the intermolecular hydrogen bonding between Apixaban molecules caused recrystallization of Apixaban (FIGS. 2 and 3).

The recrystallization of Apixaban occurs depending on the drug concentration, and when Apixaban is dissolved in dichloromethane at a concentration of 10 mg/mL or more, the recrystallization occurs. Considering the single dosage of Apixaban-loaded microsphere and its drug contents, it is difficult to prepare the Apixaban-loaded microspheres using the dispersed phase which has the 10 mg/mL or less concentrations of Apixaban.

As used herein, the term “pharmaceutically acceptable salt” refers to a salt in the form that can be pharmaceutically used, among the salts, which are the substances having cations and anions coupled by electrostatic interaction. Typically, it may include metal salts, organic base salts, inorganic acid salts, organic acid salts, basic or acidic amino acid salts, etc. Examples of the metal salts may include alkali metal salts (sodium salts, potassium salts, etc.), alkaline earth metal salts (calcium salts, magnesium salts, barium salts, etc.), or aluminum salts; examples of the salts with organic bases may include salts with triethylamine, pyridine, picoline, 2,6-lutidine, ethanolamine, diethanolamine, triethanolamine, cyclohexylamine, dicyclohexylamine, N,N′-dibenzyl ethylenediamine, etc.; examples of the inorganic acid salts may include hydrochloric acid, hydrobromic acid, nitric acid, sulfuric acid, phosphoric acid, etc.; examples of the organic acid salts may include formic acid, acetic acid, trifluoroacetic acid, phthalic acid, fumaric acid, oxalic acid, tartaric acid, maleic acid, citric acid, succinic acid, methanesulfonic acid, benzenesulfonic acid, p-toluenesulfonic acid, etc.; examples of the basic amino acid salts may include arginine, lysine, ornithine, etc.; and examples of the acidic amino acid salts include aspartic acid, glutamic acid, etc.

The composition of dispersed phase for the preparation of Apixaban-loaded microspheres of the present disclosure may contain Apixaban or a pharmaceutically acceptable salt thereof in an amount of 10% to 50% by weight relative to the biocompatible polymer, but the amount is not limited thereto.

If Apixaban or a pharmaceutically acceptable salt thereof is contained in an amount less than 10% by weight relative to the biocompatible polymer, a small amount of Apixaban is contained in the finally obtained microspheres, and thus the amount of microspheres to be administered in vivo is increased; accordingly, it may be difficult to be used clinically. In contrast, if Apixaban or a pharmaceutically acceptable salt thereof is contained in an amount greater than 50% by weight relative to the biocompatible polymer, it may not be possible to inhibit the initial burst release of Apixaban from the microspheres.

As used herein, the term “biocompatible polymer” refers to a polymer whose in vivo safety has been ensured and which does not cause high cytotoxicity and inflammatory responses when administered in vivo, and it is also simply referred to herein as a polymer.

The biocompatible polymer used in the present disclosure may be specifically polyester, and more specifically, the polyester may be any one or more selected from the group consisting of polylactic-co-glycolic acid (PLGA), polylactic acid (PLA), and polycaprolactone (PCL), but is not limited thereto.

In one specific embodiment of the present disclosure, it was confirmed that when Apixaban and the polymer are dissolved in dichloromethane simultaneously, polymer-Apixaban precipitates were formed (FIGS. 4 and 5), and it can be interpreted that the hydrogen bonds were formed between the polymer and Apixaban.

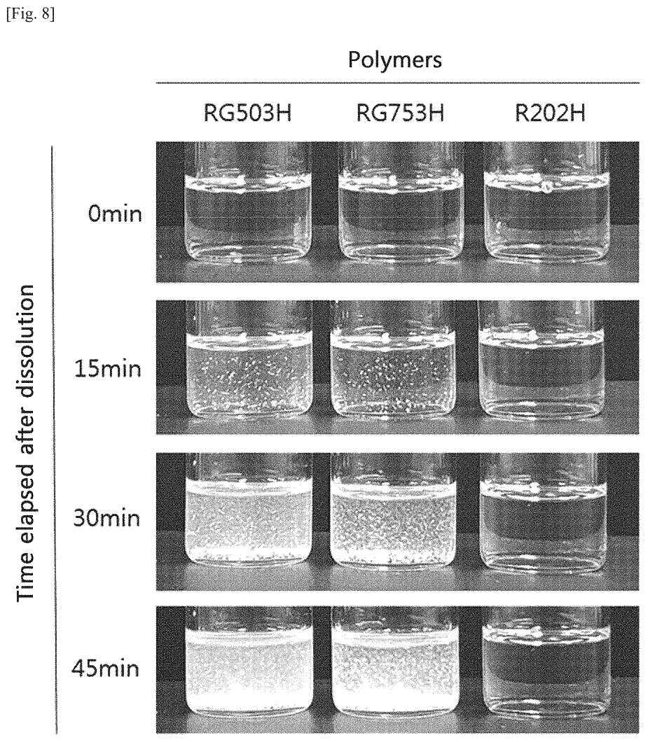

Additionally, in one specific embodiment of the present disclosure, it was confirmed that the rate, at which the polymer-Apixaban precipitates were formed, varied according to the type of polymers. Specifically, in the case of PLGA, the formation of polymer-Apixaban precipitates was promoted as the ratio of glycolide units increased, and in the case of PLA, almost no polymer-Apixaban precipitates were formed (FIG. 8). It can be interpreted that PLA was only composed of the lactide units, such that the methyl group of the lactide inhibited the formation of hydrogen bonds between the polymer and the Apixaban.

Due to the formation of such polymer-Apixaban precipitates, it is difficult to introduce the compositions of dispersed phase containing only the polymers, Apixaban, and halogen organic solvents into the manufacturing process for the Apixaban-loaded microsphere.

In the present disclosure, the polymer has an average ratio of lactide to glycolide in the polylactic-co-glycolic acid (PLGA) of 50:50 to 95:5, specifically 50:50 to 75:25, but the average ratio is not limited thereto.

In one specific embodiment of the present disclosure, as the ratio of the glycolide unit in PLGA increases, the initial drug release is inhibited, while as the ratio of the lactide unit increases, the initial drug release is promoted. It was confirmed that when the polymer having a specific glycolide-lactide ratio was used as a single polymer, the rate of initial drug release could be reduced to less than 5% under the conditions where the average ratio of lactide to glycolide is 75:25 to 50:50 (Table 3).

In the present disclosure, the polylactic acid may be used as an initial drug release promoter, and the polycaprolactone may be used as an initial drug release inhibitor.

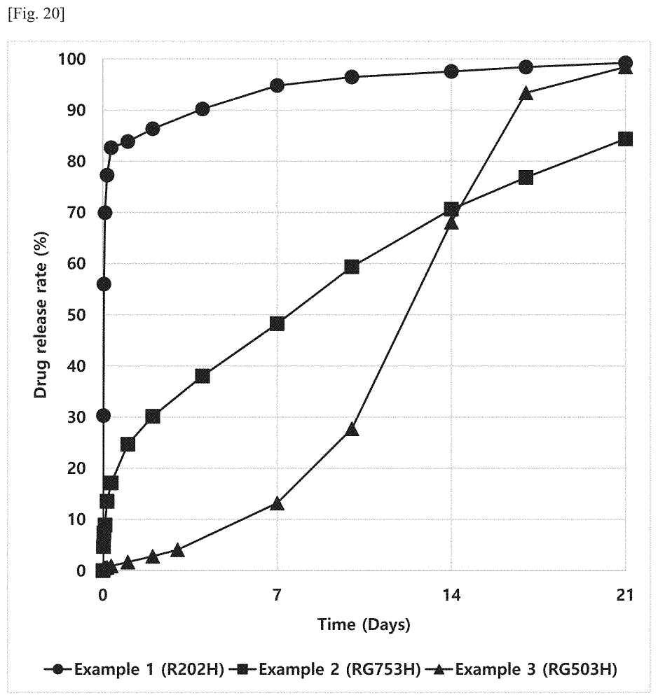

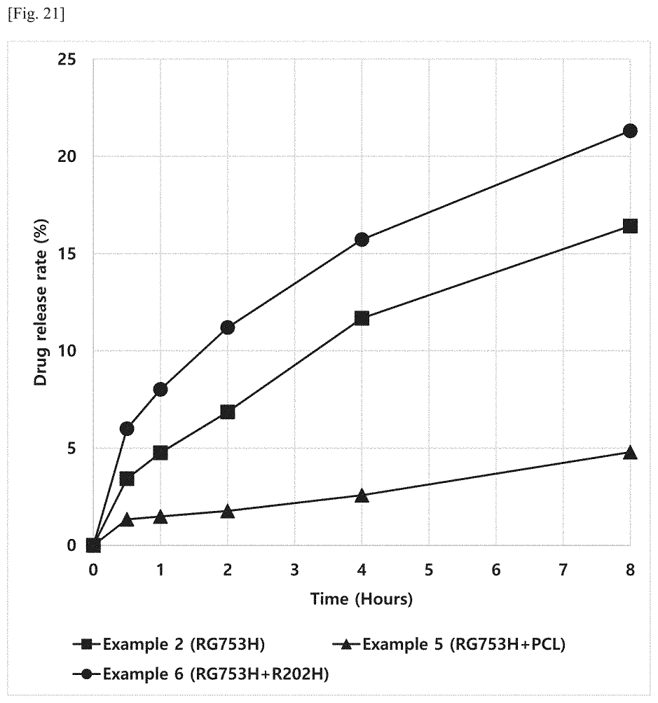

In one specific embodiment of the present disclosure, it was confirmed that PCL having no lactide residues inhibited initial drug release and thus could be used as an initial drug release inhibitor, and that PLA, which is composed only of the lactide units, promoted initial drug release (FIG. 20).

The composition of dispersed phase for the preparation of Apixaban-loaded microspheres may contain the biocompatible polymer in an amount of 5 w/v % to 30 w/v % relative to the halogen organic solvent, but is not limited thereto.

If the biocompatible polymer is contained in an amount less than 5 w/v % relative to the halogen organic solvent, and the microfluidic method is used as an example of a preparation method for microspheres, the injection time may be prolonged as the volume of the composition increases so as to use the same amount of the polymer. Also, the low drug encapsulation efficiency and the inefficient solvent removing could be occurred due to the reduced viscosity of the dispersed phase. In contrast, if the biocompatible polymer is contained in an amount greater than 30 w/v % relative to the halogen organic solvent, the viscosity may become exceedingly high, thereby imposing constraints on the preparation of microspheres.

The composition of dispersed phase for the preparation of Apixaban-loaded microspheres may include a fatty acid or triglyceride for the preparation of biocompatible polymer-based Apixaban-loaded microspheres, which facilitates the stabilization of the composition (i.e., inhibition of drug crystallization and formation of polymer-Apixaban precipitates) and stable encapsulation of drug into the microspheres, and includes of Apixaban in high contents.

In the present disclosure, the fatty acid or triglyceride can be used without limitation as long as it i) is pharmaceutically acceptable, ii) has a functional group capable of forming a hydrogen bond with Apixaban, iii) shows a high solubility in a halogen organic solvent, while having no impact on the effect of the present disclosure.

As used herein, the term “fatty acid” refers to a compound having a saturated or unsaturated aliphatic chain and refers to a compound having at least one carboxyl group. The fatty acid can be used in the present disclosure because it i) is pharmaceutically acceptable, ii) has a carboxyl group capable of forming a hydrogen bond with Apixaban, iii) shows a high solubility in a halogen organic solvent. Specifically, the fatty acid may be a C12-18 fatty acid having one or more carboxyl groups with a melting point of 35° C. or higher, which is minimum temperature for volatilizing an organic solvent when preparing microspheres, more specifically, it may be stearic acid, palmitic acid, or lauric acid, and even more specifically stearic acid or lauric acid, but is not limited thereto.

As used herein, the term “triglyceride” refers to a compound formed with three fatty acids and glycerol via an ester bond. The triglyceride can be used in the present disclosure because it i) is pharmaceutically acceptable, ii) has an ester group capable of forming a hydrogen bond with Apixaban, iii) shows a high solubility in a halogen organic solvent. Specifically, the triglyceride may be one formed with three fatty acids having at least 10 carbon atoms, which is in a solid form at room temperature, and glycerol via an ester bond, and more specifically, it may be glyceryl tridecanoate, glyceryl triundecanoate, glyceryl tridodecanoate, glyceryl trimyristate, glyceryl tripalmitate, or glyceryl tristearate. More specifically, it may be glyceryl tridodecanoate having a higher melting point than 35° C., which is a minimum temperature for volatilizing an organic solvent when preparing microspheres, but is not limited thereto.

The composition of dispersed phase for the preparation of Apixaban-loaded microspheres contains the fatty acid in a molar ratio of more than 1 time to less than 5 times relative to Apixaban and may be one contained in an amount of 50% by weight or less relative to the biocompatible polymer. Specifically, the composition of dispersed phase for the preparation of Apixaban-loaded microspheres contains the fatty acid in a molar ratio of more than 1 time to less than 5 times, more than 1 time to less than 4 times, more than 1 time to less than 3 times, more than 1 time to 2 times or less, more than 1.25 times to less than 5 times, more than 1.25 times to less than 4 times, more than 1.25 times to less than 3 times, more than 1.25 times to 2 times or less, 1.5 times or more to less than 5 times, 1.5 times or more to less than 4 times, 1.5 times or more to less than 3 times, and more specifically 1.5 times or more to 2 times or less, but is not limited thereto.

The composition of dispersed phase for the preparation of Apixaban-loaded microspheres contains the triglyceride in a molar ratio of more than 0.3 times to less than 1.6 times relative to Apixaban and in an amount of 50% by weight or less relative to the biocompatible polymer. Specifically, the composition of dispersed phase for the preparation of Apixaban-loaded microspheres contains the triglyceride in a molar ratio of more than 0.3 times to less than 1.6 times, more than 0.3 times to less than 1.3 times, more than 0.3 times to less than 1 time, more than 0.3 times to 0.7 times or less, more than 0.4 times to less than 1.6 times, more than 0.4 times to less than 1.3 times, more than 0.4 times to less than 1 time, more than 0.4 times to 0.7 times or less, 0.5 times or more to less than 1.6 times, 0.5 times or more to less than 1.3 times, 0.5 times or more to less than 1 time, and more specifically 0.5 times or more to 0.7 times or less, but is not limited thereto. Since the triglyceride is a compound containing three fatty acids, the effect of the present disclosure can be achieved even when it is used about one third molar ratio relative to the fatty acid.

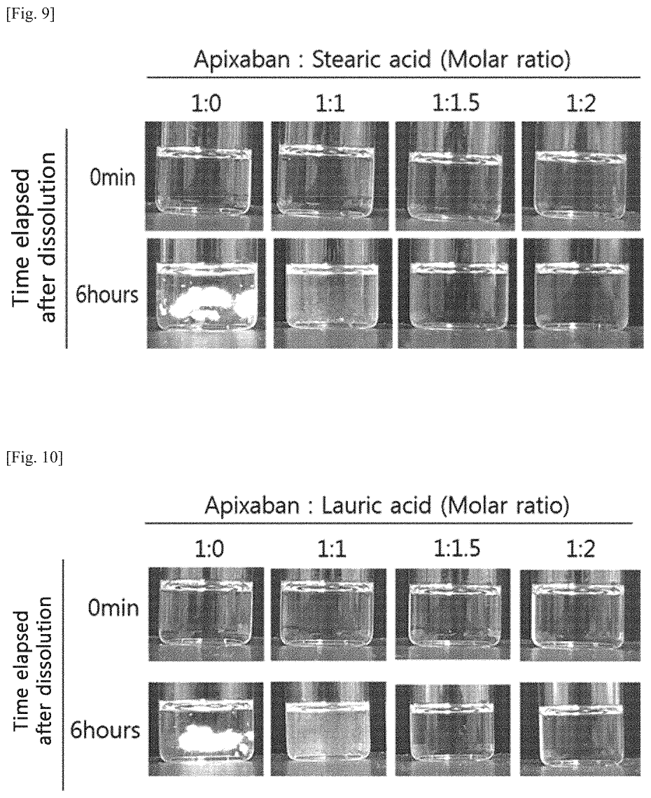

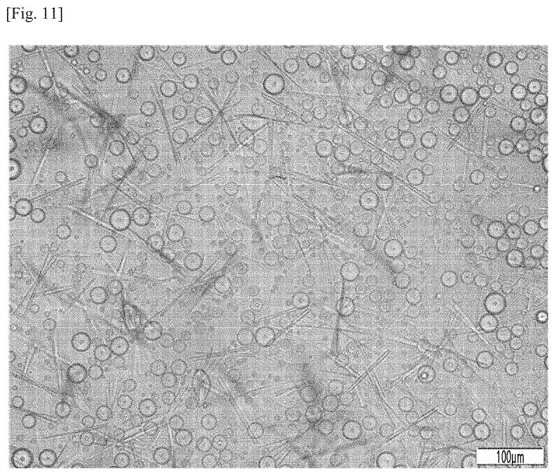

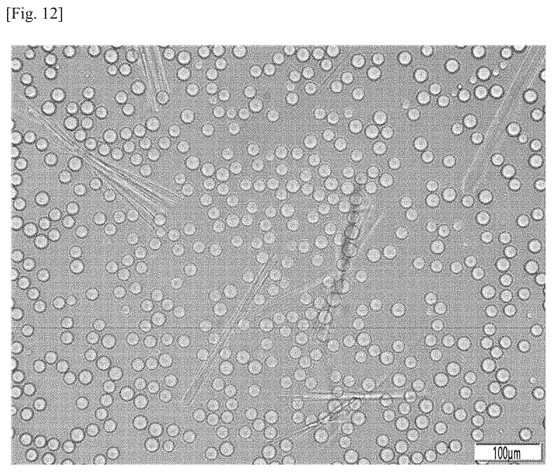

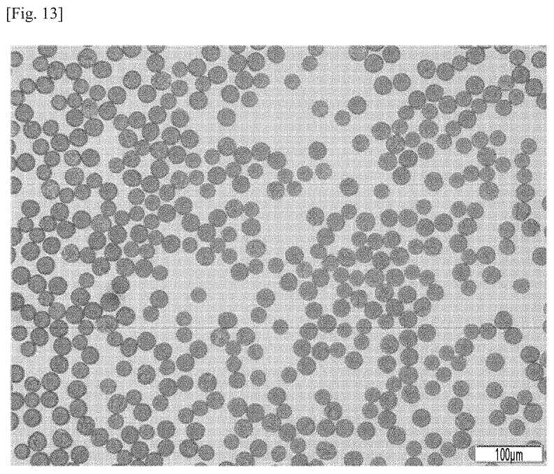

In one specific embodiment of the present disclosure, it was confirmed that when the fatty acid corresponding to the molar ratio of more than 1 time relative to Apixaban was added, a stable dispersed phase in which no precipitates were generated was formed (FIGS. 9 and 10).

If the fatty acid or triglyceride is contained in an amount greater than 50% by weight relative to the biocompatible polymer, the hardness of the microspheres may be reduced upon preparation of the microsphere, so that non-spherical particles may be prepared. The decrease in hardness and irregularity in shape of the microspheres may cause quality problems such as a decrease in physicochemical stability and a change in drug release rate.

As used herein, the term “halogen organic solvent” refers to an organic solvent containing a halogen group element, such as F, Cl, Br, or I. In the case of Apixaban, unlike other common hydrophobic drugs, Apixaban has a very low solubility in non-halogen organic solvents, and accordingly, non-halogen organic solvents cannot be used in the preparation of Apixaban microspheres.

In the present disclosure, the halogen organic solvent can be used in the preparation of microspheres as long as it does not have an impact on the effect of the present disclosure and is not limited by its type. Specifically, the halogen organic solvent may be dichloromethane (CH2Cl2), chloroform (CHCl3), carbon tetrachloride (CCl4), and more specifically may be dichloromethane, but is not limited thereto.

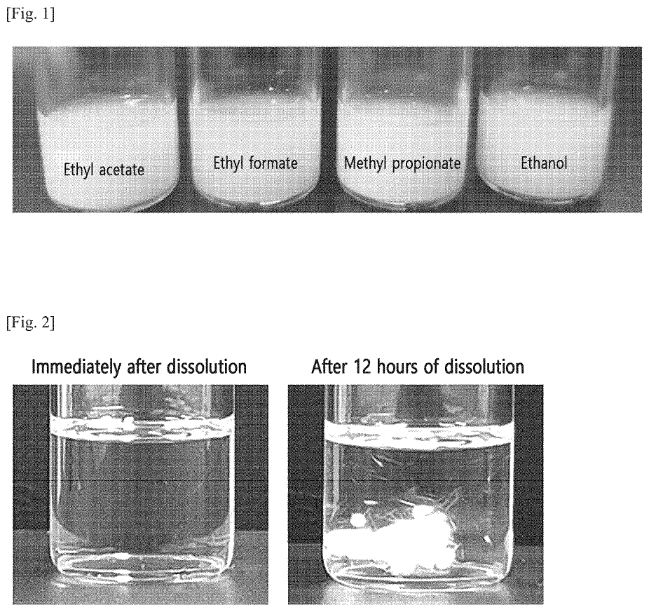

In one specific embodiment of the present disclosure it was confirmed that Apixaban was not dissolved in non-halogen organic solvents, such as ethyl acetate, ethyl formate, methyl propionate, and ethanol, but was temporarily dissolved in dichloromethane, which is a halogen organic solvent (FIGS. 1 and 2).

As used herein, the term “composition of dispersed phase for the preparation of microspheres” refers to a compound of dispersed phase which is used for the purpose of preparing microspheres, and is also simply referred to herein as a dispersed phase. The term “dispersed phase” refers to a composition for constituting an inner water phase in the case of microspheres in a water-in-oil phase, a composition for constituting an inner oil phase in the case of microspheres in an oil-in-water phase, and a composition for constituting a water-in-oil emulsion or primary emulsion in the case of microspheres in a water-in-oil-in-water phase, and thus refers to an inner phase excluding the outer phase of the composition for the preparation of microspheres, i.e., a mixture in the form in which a drug and a polymer are dissolved or dispersed.

Additionally, the composition of dispersed phase for the preparation of Apixaban-loaded microspheres may be referred to as a composition of dispersed phase for the preparation of Apixaban-loaded microspheres in the form of an injection for sustained release.

Another aspect of the present disclosure provides a biocompatible polymer-based Apixaban-loaded microsphere. Specifically, another aspect of the present disclosure may provide a biocompatible polymer-based Apixaban-loaded microsphere in the form of an injection for sustained release.

In particular, the “Apixaban” and “biocompatible polymer” are as described above.

As used herein, the term “biocompatible polymer-based Apixaban-loaded microsphere” refers to a microsphere, in which Apixaban is encapsulated and which is prepared using a biocompatible polymer, and may be also simply referred to as Apixaban-loaded microspheres, Apixaban microspheres, or microspheres. The microsphere is not limited by the type of polymers, and any microsphere falls within the scope of the present disclosure as long as Apixaban can be encapsulated in the microsphere, which is prepared using a biocompatible polymer.

If Apixaban is dissolved in dichloromethane, which is a solvent generally used for the preparation of microspheres, the drug is reprecipitated in the solution over time, and thus cannot be produced in a large scale for the use in industrial applications, and also, the drug rapidly forms crystals upon exposure or dispersion to an aqueous phase, and therefore, Apixaban has never been developed in the form of microspheres. In this regard, it is significantly meaningful from the viewpoint that the biocompatible polymer-based Apixaban-loaded microspheres have been developed for the first time by the present inventors.

The biocompatible polymer-based Apixaban-loaded microspheres of the present disclosure may include i) Apixaban or a pharmaceutically acceptable salt thereof; ii) a biocompatible polymer; and iii) a fatty acid or triglyceride.

The biocompatible polymer-based Apixaban-loaded microspheres of the present disclosure may be prepared using the composition of dispersed phase for the preparation of Apixaban-loaded microspheres.

In particular, the terms “biocompatible polymer”, “fatty acid”, “triglyceride”, “composition of dispersed phase for the preparation of microspheres”, and “dispersed phase” are as described above.

In the preparation of the Apixaban-loaded microspheres using the composition of dispersed phase for the preparation of Apixaban-loaded microspheres, the preparation method thereof is not limited as long as the Apixaban-loaded microspheres are prepared. Specifically, the Apixaban-loaded microspheres may be prepared using a solvent evaporation method, a spray drying method, a solvent extraction method, or a microfluidic method, and more specifically a microfluidic method, but the method is not limited thereto.

The biocompatible polymer-based Apixaban-loaded microspheres of the present disclosure may contain Apixaban in an amount of 5% to 30% by weight. Specifically, it may contain Apixaban in an amount of 5% to 30% by weight, 8% to 28% by weight, 10% to 25% by weight, 12% to 22% by weight, and more specifically 15% to 20% by weight, but the amount is not limited thereto.

In one specific embodiment of the present disclosure, it was confirmed that the microspheres of Examples 1 to 6 provided by the present invention contained Apixaban in high contents in an amount of 15% to 20% by weight (Table 2).

The biocompatible polymer-based Apixaban-loaded microspheres of the present disclosure may release Apixaban by 5% or less in the initial 30 minutes. With respect to the average ratio of lactide to glycolide in the biocompatible polymer or the mixing ratio of the biocompatible polymer, which is a factor involved in the initial drug release of Apixaban, all combinations of these factors are within the scope of the present disclosure as long as they can release Apixaban by 5% or less in the initial 30 minutes.

In one specific example of the present disclosure, it was confirmed that Examples 2 to 5 could release Apixaban by 5% or less within the initial 30 minutes (Table 3).

In the present disclosure, the release may be controlled by the average ratio of lactide to glycolide in the biocompatible polymer.

There is a tendency of suppressing the initial burst release of Apixaban when the microsphere is composed by the polymer which has an increased number of functional groups capable of forming hydrogen bonds with Apixaban in a neutral environment. Unlike the general release patterns of hydrophobic drugs in microsphere, the release of Apixaban is suppressed as the polymer having a higher ratio of glycolide units in PLGA is used, and PLA has a faster initial drug release as compared to PLGA. This may be interpreted that the methyl group of the lactide inhibits the formation of hydrogen bonds between the Apixaban and the polymer.

In one specific embodiment of the present disclosure, it was confirmed that when the polymer having a specific glycolide-lactide ratio was used as a single polymer, the initial drug release could be reduced to less than 5% under the conditions where the average ratio of lactide to glycolide was 50:50 (Example 3) to 75:25 (Example 2) (Table 3).

In the present disclosure, the release may be controlled by the mixing ratio of the biocompatible polymer.

In the same principle, PCL which does not contain a methyl group in the chemical structure of the polymer not only facilitates the formation of hydrogen bonds with Apixaban, but may also contribute to suppress the initial burst release.

Thus, since the initial drug release rate is determined according to the mixing ratio of the polymers when a mixture of the polymers is used, the mixing ratio of the polymers is not particularly limited, and an appropriate mixing ratio of the polymers may be selected according to the initial drug release rate to be applied.

In one specific embodiment of the present disclosure, it was confirmed that when PCL, which is a polymer without lactide units was used together with PLGA (Example 5), the initial drug release could be suppressed, and that when PLA, which is composed only of the lactide units, was used together with PLGA (Example 6), the initial drug release could be promoted (FIG. 20).

Still another aspect of the present disclosure provides a pharmaceutical composition including the biocompatible polymer-based Apixaban-loaded microspheres.

In particular, the terms “Apixaban” and “biocompatible polymer-based Apixaban-loaded microspheres” are as described above.

The pharmaceutical composition of the present disclosure may be for sustained release of Apixaban.

As used herein, the term “sustained release” refers to releasing the drug for a long time in vivo by controlling the release mechanism of the drug. Specifically, in the present invention, it may refer to the inhibition of the initial burst release, but is not limited thereto.

The pharmaceutical composition of the present disclosure may be used for the prevention or treatment of all target diseases, specifically nonvalvular atrial fibrillation, deep vein thrombosis, pulmonary embolism, etc., for which Apixaban may have a preventive or treatment effect, but the disease is not limited thereto.

In addition, it may be used as an anticoagulant, but is not limited thereto.

The pharmaceutical composition of the present disclosure may further include an excipient or diluent, in addition to the Apixaban-loaded microspheres.

Specifically, excipients and diluents that may be included in the pharmaceutical composition include cryoprotectants such as lactose, dextrose, sucrose, sorbitol, mannitol, erythritol, maltitol, etc., thickeners such as starch, alginate, gelatin, cellulose, methylcellulose, carboxymethylcellulose, etc., pharmaceutically usable pH buffers, surfactants, or water.

The pharmaceutical composition may be formulated into an injection for subcutaneous or intramuscular administration, but is not limited thereto.

### MODE FOR CARRYING OUT THE PRESENT DISCLOSURE

The present disclosure will be described in more detail by way of Examples. However, these Examples are given for illustrative purposes only, and the scope of the disclosure is not intended to be limited to or by these Examples.

### EXPERIMENTAL EXAMPLE 1

### Stability of Compositions of Dispersed Phase for Preparation of Apixaban-Loaded Microspheres

### Experimental Example 1-1

### Dissolution of Apixaban in Non-Halogen Organic Solvents

25 mg of Apixaban was added to 1 mL of each of ethyl acetate, ethyl formate, methyl propionate, and ethanol and stirred. As a result, it was confirmed that Apixaban was not dissolved in non-halogen organic solvents (FIG. 1).

From these results, it was found that the non-halogen organic solvents could not be used as the solvent for preparing Apixaban microspheres.

### Experimental Example 1-2

### Dissolution of Apixaban in Halogen Organic Solvent

25 mg of Apixaban was dissolved in 1 mL of dichloromethane, and the mixture was photographed after 12 hours. As a result, it was found that Apixaban was completely dissolved in the beginning, but after a certain time, crystals were formed in dichloromethane (FIG. 2).

Additionally, the crystals were observed under an optical microscope, and as a result, it was found that needle-like structures were formed (FIG. 3).

From these results, it was found that even if Apixaban was temporarily dissolved in dichloromethane, it was recrystallized in the solvent over time due to a high crystallinity of Apixaban.

### Experimental Example 1-3

### Stability of General Composition of Dispersed Phase for Preparation of Apixaban-Loaded Microspheres

100 mg of PLGA RG503H and 25 mg of Apixaban were dissolved in 1 mL of dichloromethane, and the mixture was photographed after 0 minutes, 15 minutes, and 30 minutes. As a result, it was confirmed that Apixaban was completely dissolved in the beginning, but after a certain time, polymer-Apixaban precipitates were formed (FIG. 4).

Additionally, the polymer-Apixaban precipitates were observed under an optical microscope, and as a result, it was found that the needle-like structures of the drug and the polymer were aggregated in the precipitates (FIG. 5).

From these results, it was confirmed that the general composition of dispersed phase (drug+polymer+halogen organic solvent) for the preparation of Apixaban-loaded microspheres could not be used alone in the preparation of microspheres due to low stability.

### Experimental Example 1-4

### Stability of Composition of Dispersed Phase for Preparation of Apixaban-Loaded Microspheres According to Addition of Fatty Acids

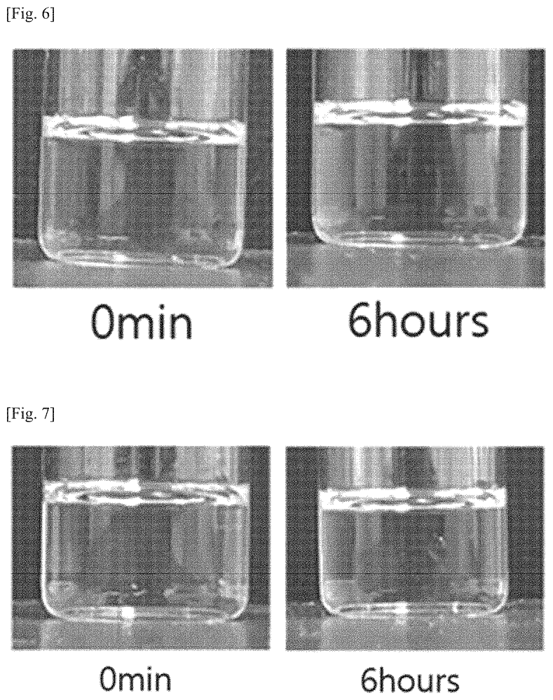

100 mg of PLGA RG503H, 25 mg of Apixaban, and 25 mg of stearic acid were dissolved in 1 mL of dichloromethane, and the mixture was photographed after 0 minutes and 6 hours. As a result, it was confirmed that a stable dispersed phase, in which no precipitates were generated, was formed (FIG. 6).

Additionally, 100 mg of PLGA RG503H, 25 mg of Apixaban, and 17.3 mg of lauric acid were dissolved in 1 mL of dichloromethane, and the mixture was photographed after 0 minutes and 6 hours. As a result, it was confirmed that a stable dispersed phase, in which precipitates were not generated, was formed (FIG. 7).

From these results, it was found that the compositions of the dispersed phase for the preparation of Apixaban-loaded microspheres, to which the fatty acids were added, provided by the present disclosure showed the improved stability and thus could be used in the preparation of the microspheres.

### EXPERIMENTAL EXAMPLE 2

### Stability of Compositions of Dispersed Phase for Preparation of Apixaban-Loaded Microspheres According Types of Polymers

100 mg of polymers (PLGA RG503H, PLGA RG753H, and PLA R202H) and 25 mg of Apixaban were dissolved in 1 mL of dichloromethane, and the mixture was photographed at 15-minute intervals for 45 minutes. As a result, it was found that an excess amount of polymer-Apixaban precipitates, in which the polymers and Apixaban were aggregated, was formed as the ratio of the glycolide units in the PLGA increased, and in the case of PLA R202H, which is composed only of the lactide units, no polymer-Apixaban precipitates were formed (FIG. 8).

This may be interpreted that the methyl group of the lactide units inhibited the formation of hydrogen bonds between the polymer and the Apixaban.

### EXPERIMENTAL EXAMPLE 3

### Stability of Compositions of Dispersed Phase for Preparation of Apixaban-Loaded Microspheres according Molar Ratio of Apixaban and Fatty Acids

### Experimental Example 3-1

### Stability of Compositions of Dispersed Phase for Preparation of Apixaban-Loaded Microspheres according Molar Ratio of Apixaban and Stearic Acid

100 mg of PLGA RG503H, 25 mg Apixaban, and stearic acid at different concentrations (molar ratio of stearic acid to Apixaban was 1:0, 1:1, 1:1.5, 1:2) were dissolved in 1 mL of dichloromethane, and the mixture was photographed after 6 hours. As a result, it was confirmed that when the stearic acid was added in the molar ratio of more than 1 time relative to Apixaban, a stable dispersed phase, in which no precipitates were generated, was formed (FIG. 9).

### Experimental Example 3-2

### Stability of Compositions of Dispersed Phase for Preparation of Apixaban-Loaded Microspheres according Molar Ratio of Apixaban and Lauric Acid

100 mg of PLGA RG503H, 25 mg Apixaban, and lauric acid at different concentrations (molar ratio of lauric acid to Apixaban was 1:0, 1:1, 1:1.5, 1:2) were dissolved in 1 mL of dichloromethane, and the mixture was photographed after 6 hours. As a result, it was confirmed that when the lauric acid was added in the molar ratio of more than 1 time relative to Apixaban, a stable dispersed phase, in which no precipitates were generated, was formed (FIG. 10).

### EXPERIMENTAL EXAMPLE 4

### Preparation of Apixaban Microspheres using General Composition of Dispersed Phase for Preparation of Apixaban-Loaded Microspheres

### Experimental Example 4-1

### Preparation of Apixaban-Loaded Microspheres using Solvent Evaporation Method (Comparative Example 1)

A dispersed phase was prepared by simultaneously dissolving 25 mg of Apixaban and 100 mg of PLA R202H in 1 mL of dichloromethane, and then the resultant was dispersed in a 1% poly vinyl alcohol (PVA) solution using a high shear mixer (Silverson, L5M-A), which was stirred at 1,500 rpm. In particular, Apixaban rapidly precipitated into the aqueous phase to form needle-like crystals simultaneously with the start of the dispersion (FIG. 11).

From these results, it was found that when the general composition of dispersed phase for the preparation of Apixaban-loaded microspheres (drug+polymer+halogen organic solvent) was used, Apixaban microspheres could not be prepared by the solvent evaporation method.

### Experimental Example 4-2

### Preparation of Apixaban-Loaded Microspheres using Microfluidic Method (Comparative Example 2)

A dispersed phase was prepared by simultaneously dissolving 25 mg of Apixaban and 100 mg of PLA R202H in 1 mL of dichloromethane, and then the resultant was injected into a microfluidic chip (Dolomite, 3D focusing hydrophilic chip) at a flow rate of 0.01 mL/min. In particular, a 1% PVA solution was used as the continuous phase, which was injected simultaneously with the dispersed phase at a flow rate of 0.1 mL/min, and liquid drops formed inside the microfluidic chip were obtained in the 1% PVA solution, which was stirred at 150 rpm. Subsequently, the obtained liquid drops were observed under an optical microscope, and as a result, it was confirmed that needle-like drug crystals were formed in large amounts (FIG. 12).

From these results, it was found that when the general composition of dispersed phase for the preparation of Apixaban-loaded microspheres (drug+polymer+halogen organic solvent) was used, Apixaban microspheres could not be prepared by the microfluidic method.

In order to separate the microspheres from the drug crystals as much as possible, the mixture was washed three times with pure water using a 75 μm mesh sieve. The separated microspheres were obtained using a membrane filter, followed by freeze-drying for 2 days to obtain dried microspheres.

### EXPERIMENTAL EXAMPLE 5

### Preparation of Apixaban Microspheres using Composition of Dispersed Phase for Preparation of Apixaban-Loaded Microspheres of the Present Disclosure

### Experimental Example 5-1

### Preparation of Apixaban-Loaded Microspheres (Example 1)

A dispersed phase was prepared by simultaneously dissolving 25 mg of Apixaban, 100 mg of PLA R202H, and 25 mg of stearic acid in 1 mL of dichloromethane, and then the resultant was injected into a microfluidic chip (Dolomite, 3D focusing hydrophilic chip) at a flow rate of 0.01 mL/min. In particular, a 1% PVA solution was used as the continuous phase, which was injected simultaneously with the dispersed phase at a flow rate of 0.1 mL/min, and liquid drops formed inside the microfluidic chips were obtained in the 1% PVA solution, which was stirred at 150 rpm. Subsequently, the thus-obtained microsphere liquid drops were observed under an optical microscope, and as a result, no drug precipitated from the microsphere liquid drops was observed (FIG. 13).

The microsphere liquid drops were further stirred at 35° C. for 2 hours to volatilize the organic solvent. After the organic solvent was removed, the microspheres were cured using a membrane filter, and then dried for 2 days by freeze-drying to obtain dried microspheres.

### Experimental Example 5-2

### Preparation of Apixaban-Loaded Microspheres (Example 2)

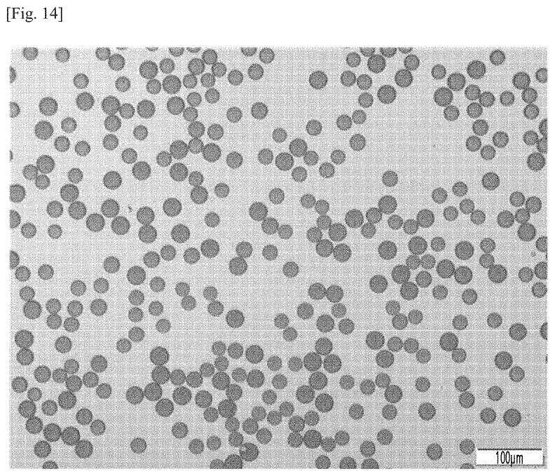

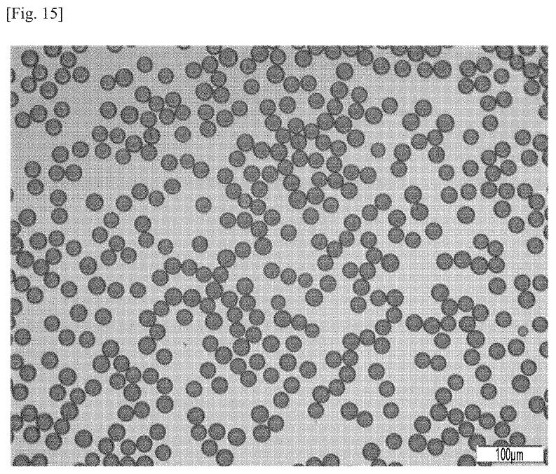

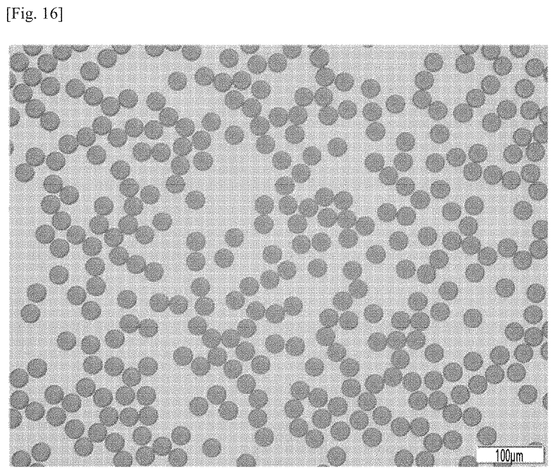

A dispersed phase was prepared by simultaneously dissolving 25 mg of Apixaban, 100 mg of PLGA RG753H, and 25 mg of stearic acid in 1 mL of dichloromethane, and then the resultant was injected into a microfluidic chip (Dolomite, 3D focusing hydrophilic chip) at a flow rate of 0.01 mL/min. In particular, a 1% PVA solution was used as the continuous phase, which was injected simultaneously with the dispersed phase at a flow rate of 0.1 mL/min, and liquid drops formed inside the microfluidic chips were obtained in the 1% PVA solution, which was stirred at 150 rpm. Subsequently, the thus-obtained microsphere liquid drops were observed under an optical microscope, and as a result, no drug precipitated from the microsphere liquid drops was observed (FIG. 14).

The microsphere liquid drops were further stirred at 35° C. for 2 hours to volatilize the organic solvent. After removing the organic solvent, the microspheres were cured using a membrane filter, and then dried for 2 days by freeze-drying to obtain dried microspheres.

### Experimental Example 5-3

### Preparation of Apixaban-Loaded Microspheres (Example 3)

A dispersed phase was prepared by simultaneously dissolving 25 mg of Apixaban, 100 mg of PLGA RG503H, and 25 mg of stearic acid in 1 mL of dichloromethane, and then the resultant was injected into a microfluidic chip (Dolomite, 3D focusing hydrophilic chip) at a flow rate of 0.01 mL/min. In particular, a 1% PVA solution was used as the continuous phase, which was injected simultaneously with the dispersed phase at a flow rate of 0.1 mL/min, and liquid drops formed inside the microfluidic chips were obtained in the 1% PVA solution, which was stirred at 150 rpm. Subsequently, the thus-obtained microsphere liquid drops were observed under an optical microscope, and as a result, no drug precipitated from the microsphere liquid drops was observed (FIG. 15).

The microsphere liquid drops were further stirred at 35° C. for 2 hours to volatilize the organic solvent. After removing the organic solvent, the microspheres were cured using a membrane filter, and then dried for 2 days by freeze-drying to obtain dried microspheres.

### Experimental Example 5-4

### Preparation of Apixaban-Loaded Microspheres (Example 4)

A dispersed phase was prepared by simultaneously dissolving 25 mg of Apixaban, 100 mg of PLGA RG503H, and 25 mg of lauric acid in 1 mL of dichloromethane, and then the resultant was injected into a microfluidic chip (Dolomite, 3D focusing hydrophilic chip) at a flow rate of 0.01 mL/min. In particular, a 1% PVA solution was used as the continuous phase, which was injected simultaneously with the dispersed phase at a flow rate of 0.1 mL/min, and liquid drops formed inside the microfluidic chips were obtained in the 1% PVA solution, which was stirred at 150 rpm. Subsequently, the thus-obtained microsphere liquid drops were observed under an optical microscope, and as a result, no drug precipitated from the microsphere liquid drops was observed (FIG. 16).

The microsphere liquid drops were further stirred at 35° C. for 2 hours to volatilize the organic solvent. After the organic solvent was removed, the microspheres were cured using a membrane filter, and then dried for 2 days by freeze-drying to obtain dried microspheres.

### Experimental Example 5-5

### Preparation of Apixaban-Loaded Microspheres (Example 5)

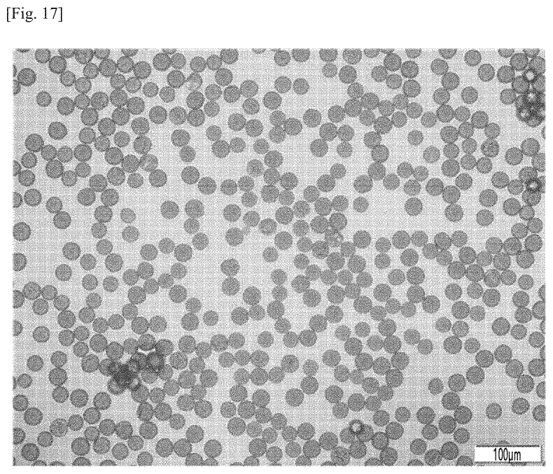

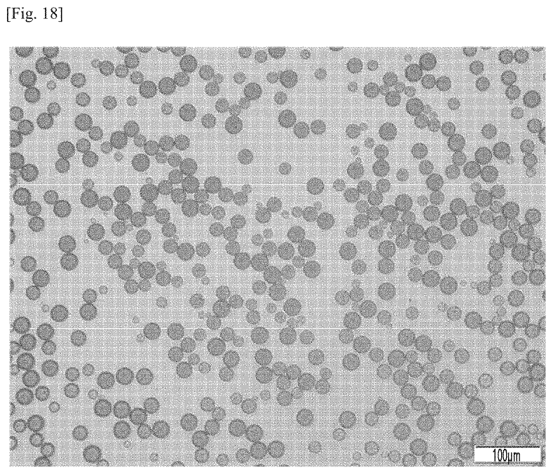

A dispersed phase was prepared by simultaneously dissolving 25 mg of Apixaban, 90 mg of PLGA RG753H, 10 mg of PCL (average molecular weight of 45,000 g/mol), and 25 mg of stearic acid in 1 mL of dichloromethane, and then the resultant was injected into a microfluidic chip (Dolomite, 3D focusing hydrophilic chip) at a flow rate of 0.01 mL/min. In particular, a 1% PVA solution was used as the continuous phase, which was injected simultaneously with the dispersed phase at a flow rate of 0.1 mL/min, and liquid drops formed inside the microfluidic chips were obtained in the 1% PVA solution, which was stirred at 150 rpm. Subsequently, the thus-obtained microsphere liquid drops were observed under an optical microscope, and as a result, no drug precipitated from the microsphere liquid drops was observed (FIG. 17).

The microsphere liquid drops were further stirred at 35° C. for 2 hours to volatilize the organic solvent. After removing the organic solvent, the microspheres were cured using a membrane filter, and then dried for 2 days by freeze-drying to obtain dried microspheres.

### Experimental Example 5-6

### Preparation of Apixaban-Loaded Microspheres (Example 6)

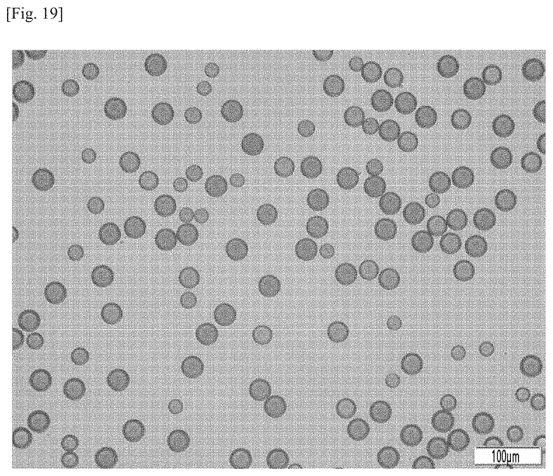

A dispersed phase was prepared by simultaneously dissolving 25 mg of Apixaban, 90 mg of PLGA RG753H, 10 mg of PLA R202H, and 25 mg of stearic acid in 1 mL of dichloromethane, and then the resultant was injected into a microfluidic chip (Dolomite, 3D focusing hydrophilic chip) at a flow rate of 0.01 mL/min. In particular, a 1% PVA solution was used as the continuous phase, which was injected simultaneously with the dispersed phase at a flow rate of 0.1 mL/min, and liquid drops formed inside the microfluidic chips were obtained in the 1% PVA solution, which was stirred at 150 rpm. Subsequently, the thus-obtained microsphere liquid drops were observed under an optical microscope, and as a result, no drug precipitated from the microsphere liquid drops was observed (FIG. 18).

The microsphere liquid drops were further stirred at 35° C. for 2 hours to volatilize the organic solvent. After the organic solvent was removed, the microspheres were cured using a membrane filter, and then dried for 2 days by freeze-drying to obtain dried microspheres.

### Experimental Example 5-7

### Preparation of Apixaban-Loaded Microspheres (Example 7)

A dispersed phase was prepared by simultaneously dissolving 2.5 mg of Apixaban, 100 mg of PLGA RG503H, 10 mg of PLA R202H, and 18.7 mg of glyceryl tridodecanoate in 1 mL of dichloromethane, and then the resultant was injected into a microfluidic chip (Dolomite, 3D focusing hydrophilic chip) at a flow rate of 0.01 mL/min. In particular, a 1% PVA solution was used as the continuous phase, which was injected simultaneously with the dispersed phase at a flow rate of 0.1 mL/min, and liquid drops formed inside the microfluidic chips were obtained in the 1% PVA solution, which was stirred at 150 rpm. Subsequently, the thus-obtained microsphere liquid drops were observed under an optical microscope, and as a result, no drug precipitated from the microsphere liquid drops was observed (FIG. 19).

The microsphere liquid drops were further stirred at 35° C. for 2 hours to volatilize the organic solvent. After the organic solvent was removed, the microspheres were cured using a membrane filter, and then dried for 2 days by freeze-drying to obtain dried microspheres.

### EXPERIMENTAL EXAMPLE 6

### Analysis of Drug Content in Apixaban-Loaded Microspheres

In order to measure the drug content of the freeze-dried microspheres corresponding to Comparative Examples 1 and 2 prepared in Experimental Example 4, and Examples 1 to 7 prepared in Experimental Example 5, 1 mg of the finally freeze-dried microspheres were dissolved in in acetonitrile and filtered using a 0.45 μm PVDF syringe filter, and then subjected to quantitative analysis using a HPLC-UV device according to the conditions shown in Table 1 below.

TABLE 1

| | |
|---|---|
| Mobile phase | Water:ACN (60:40) |
| Column | YMC-Triart C18 column, |
| | C18 (150 × 4.0 mm ID), S-5 μm |
| Flow rate | 1 mL/min |
| Column temperature | 20° C. |
| Wavelength | 281 nm |
| Injection volume | 20 μL |

The content of the drug encapsulated in the microspheres was calculated Equation (1) below.

Drug Content=Drug Concentration analyzed by HPLC (mg/mL)÷1 mg/mL×100(%) (1)

The results of analyzing the Apixaban content in the microspheres calculated by the Equation (1) are shown in Table 2.

TABLE 2

| | Compar- | Compar- | | | | | | | |
|---|---|---|---|---|---|---|---|---|---|
| | ative | ative | | | | | | | |
| | Example | Example | Example | Example | Example | Example | Example | Example | Example |
| | 1 | 2 | 1 | 2 | 3 | 4 | 5 | 6 | 7 |
| Drug | Measure- | 0.57% | 18.40 | 17.61 | 18.36 | 20.41 | 18.60 | 18.65 | 18.92 |
| Content | ment Not | | | | | | | | |
| (%) | Possible | | | | | | | | |

Specifically, in the case of Comparative Example 1, it was difficult to remove the drug crystals due to high contents of drugs crystals, and thus, it was not possible to measure the Apixaban content in the microspheres. In the case of Comparative Example 2, the microspheres obtained by removing the drug crystals as much as possible by washing three times with water using a 75 μm mesh sieve, followed by freeze-drying were used to measure the Apixaban content in the microspheres.

As a result, it was confirmed that the microspheres of Examples 1 to 7 provided by the present disclosure could contain Apixaban in high contents in an amount of 15% to 20%.

From these results, it was found that even after freeze-drying, Apixaban was well encapsulated in the microspheres provided by the present disclosure.

### EXPERIMENTAL EXAMPLE 7

### Drug Release Analysis of Apixaban-Loaded Microspheres

### Experimental Example 7-1

### Drug Release Analysis According to Types of Polymers

In order to analyze the drug release of the microspheres corresponding to Examples 1 to 6 prepared in Experimental Example 5 according to the types of polymers, 28 mg of the microspheres corresponding to Examples 1 to 7 were independently immersed in 50 mL of a phosphate buffer at pH 7.4 containing 0.2% sodium lauryl sulfate (ion strength of 154 mM), and then subjected to reciprocal shaking at 37° C. and 50 rpm using a constant-temperature water bath. After 30 minutes, 1 mL of the supernatant was collected from each solution and centrifuged at 9,000 rpm for 5 minutes. Subsequently, 0.5 mL of the supernatant was quantitatively analyzed using a HPLC-UV device, and the drug release rate was calculated by Equation (2) below.

Drug release rate=Drug Concentration in Release Sample÷Theoretical Drug Concentration upon Complete×100(%) (2)

The results of analyzing the drug release rate of the microspheres calculated by Equation (2) are shown in Table 3.

TABLE 3

| | Example 1 | Example 2 | Example 3 | Example 4 | Example 5 | Example 6 | Example 7 |
|---|---|---|---|---|---|---|---|
| Drug | 30.30 | 4.07 | 1.74 | 1.34 | 1.34 | 6.00 | 1.33 |
| release rate | | | | | | | |
| at 30 min | | | | | | | |
| (%) | | | | | | | |

### Experimental Example 7-2

### Drug Release Analysis According to Time

In order to analyze the drug release of the microspheres corresponding to Examples 1 to 3 prepared in Experimental Example 5 according to the time, 28 mg of the microspheres corresponding to Examples 1 to 3 were independently immersed in 50 mL of a phosphate buffer at pH 7.4 containing 0.2% sodium lauryl sulfate (ion strength of 154 mM), and then subjected to reciprocal shaking at 37° C. and 50 rpm using a constant-temperature water bath. At predetermined times (1, 2, 4, 7, 10, 14, 17, 21 days after 0.5, 1, 2, 4, 8 hours), 1 mL of the supernatant was collected from each solution and centrifuged at 9,000 rpm for 5 minutes. 0.5 mL of the supernatant was quantitatively analyzed using a HPLC-UV device under the same conditions as in Experimental Example 7-1, and the release rate thereof was calculated by Equation (2) above.

The results of analyzing the release rate of the microspheres calculated by Equation (2) are shown in FIG. 20.

As a result of the experiment, it was found that the drug release was inhibited as the ratio of the glycolide units in the PLGA increased, and the drug release was promoted as the ratio of the lactide units increased. Accordingly, it was confirmed that when the polymer having a specific glycolide-lactide ratio was as a single polymer, the initial drug release could be reduced to less than 5% under the conditions where the average ratio of lactide to glycolide was 50:50 (Example 3) to 75:25 (Example 2).

When PLA was used as a single polymer (Example 1; where the average ratio of lactide to glycolide is 100:0), Apixaban rapidly diffused from the microspheres to the dissolution solution at the beginning of the dissolution. As confirmed in Example 2, it can be interpreted that the methyl group of the lactide unit inhibited the formation of hydrogen bond between the polymer and Apixaban.

Therefore, as shown in FIG. 21, PCL having no lactide units (Example 5) inhibits the initial drug release and thus can be used as an initial drug release inhibitor, and PLA (Example 6), which is composed only of the lactide units, promotes the initial drug release and thus can be used as an initial drug release promoter.

While the present disclosure has been described with reference to the particular illustrative embodiments, it will be understood by those skilled in the art to which the present disclosure pertains that the present disclosure may be embodied in other specific forms without departing from the technical spirit or essential characteristics of the present disclosure. Therefore, the embodiments described above are considered to be illustrative in all respects and not restrictive. Furthermore, the scope of the present disclosure is defined by the appended claims rather than the detailed description, and it should be understood that all modifications or variations derived from the meanings and scope of the present disclosure and equivalents thereof are included in the scope of the appended claims.

1. A composition of dispersed phase for the preparation of Apixaban-loaded microspheres, the composition comprising:
i) Apixaban or a pharmaceutically acceptable salt thereof;
ii) a biocompatible polymer;
iii) a fatty acid or triglyceride; and
iv) a halogen organic solvent.

2. The composition of claim 1, wherein the Apixaban or the pharmaceutically acceptable salt thereof includes in an amount of 10% to 50% by weight relative to the biocompatible polymer.

3. The composition of claim 1, wherein the biocompatible polymer is polyester.

4. The composition of claim 3, wherein the polyester is selected from the group consisting of polylactic-co-glycolic acid (PLGA), polylactic acid (PLA), and polycaprolactone (PCL).

5. The composition of claim 4, wherein the average ratio of lactide to glycolide in the polylactic-co-glycolic acid is 50:50 to 95:5.

6. The composition of claim 1, wherein the biocompatible polymer includes in an amount of 5 w/v % to 30 w/v % relative to the halogen organic solvent.

7. The composition of claim 1, wherein the fatty acid is a C12-18 fatty acid having one or more carboxyl groups.

8. The composition of claim 7, wherein the fatty acid is stearic acid or lauric acid.

9. The composition of claim 1, wherein the triglyceride is formed with glycerol and three fatty acids having at least 10 carbon atoms via an ester bond.

10. The composition of claim 9, wherein the triglyceride is glyceryl tridodecanoate.

11. The composition of claim 1, wherein the fatty acid includes in a molar ratio of more than 1 times to less than 5 times relative to Apixaban and in an amount of 50% by weight or less relative to the biocompatible polymer.

12. The composition of claim 1, wherein the triglyceride includes in a molar ratio of more than 0.3 times to 1.6 times relative to Apixaban and in an amount of 50% by weight or less relative to the biocompatible polymer.

13. The composition of claim 1, wherein the halogen organic solvent is dichloromethane.

14. A biocompatible polymer-based Apixaban-loaded microsphere comprising:
i) Apixaban or a pharmaceutically acceptable salt thereof;
ii) a biocompatible polymer; and
iii) a fatty acid or triglyceride.

15. The microsphere of claim 14, wherein the biocompatible polymer-based Apixaban-loaded microsphere is prepared from the composition of dispersed phase for the preparation of Apixaban-loaded microspheres according to claim 1.

16. The microsphere of claim 14, wherein the biocompatible polymer-based Apixaban-loaded microsphere comprises 5% to 30% by weight of Apixaban relative to the microsphere.

17. The microsphere of claim 14, wherein the biocompatible polymer-based Apixaban-loaded microsphere releases Apixaban by 5% or less in the initial 30 minutes.

18. A pharmaceutical composition comprising the biocompatible polymer-based Apixaban-loaded microsphere of claim 14.

19. The pharmaceutical composition of claim 18, wherein the pharmaceutical composition is for sustained release of Apixaban.

20. The pharmaceutical composition of claim 18, wherein the pharmaceutical composition is formulated into an injection for subcutaneous administration or intramuscular administration.
